(19) United States
(12) Patent Application Publication
(10) Pub. No.: US 2024/0197714 A1
(43) Pub. Date: Jun. 20, 2024

(54) METHODS FOR THE ADMINISTRATION OF CERTAIN VMAT2 INHIBITORS

(71) Applicant: Neurocrine Biosciences, Inc., San Diego, CA (US)

(72) Inventors: Gordon Loewen, San Diego, CA (US); Satjit Brar, San Diego, CA (US)

(21) Appl. No.: 18/286,944

(22) PCT Filed: Apr. 14, 2022
(86) PCT No.: PCT/US2022/024848

§ 371 Date: Oct. 13, 2023

Related U.S. Application Data
(60) Provisional application No. 63/238,502, filed on Aug. 30, 2021.
(60) Provisional application No. 63/189,946, filed on May 18, 2021.
(60) Provisional application No. 63/175,379, filed on Apr. 15, 2021.

(51) Int. Cl. A61K 31/4745; A61K 9/00; A61K 9/20; A61K 9/48

(52) U.S. Cl. CPC A61K 31/4745; A61K 9/0053; A61K 9/20; A61K 9/48

(57) ABSTRACT

Provided is a method of treating a neurological or psychiatric disease or disorder in a patient in need thereof, comprising: administering a vesicular monoamine transporter 2 (VMAT2) inhibitor to the patient in need thereof wherein the patient is a CYP2D6 poor metabolizer.

Dysregulation of dopaminergic systems is integral to several central nervous system (CNS) disorders, including neurological and psychiatric diseases and disorders. These neurological and psychiatric diseases and disorders include hyperkinetic movement disorders, and conditions such as schizophrenia and mood disorders. The transporter protein vesicular monoamine transporter 2 (VMAT2) plays an important role in presynaptic dopamine release and regulates monoamine uptake from the cytoplasm to the synaptic vesicle for storage and release.

Despite the advances that have been made in this field, there remains a need for new therapeutic products useful to treatment of neurological and psychiatric diseases and disorders and other related diseases or conditions described herein. One such agent is deutetrabenazine, which is a racemic mixture of the following compounds:

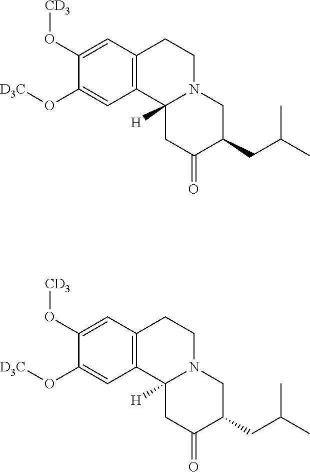 [SMILES: [2H]C([2H])([2H])Oc1cc2c(cc1OC([2H])([2H])[2H])[C@H]1CC(=O)[C@H](CC(C)C)CN1CC2 ; [2H]C([2H])([2H])Oc1cc2c(cc1OC([2H])([2H])[2H])[C@@H]1CC(=O)[C@@H](CC(C)C)CN1CC2]

A formulation of deutetrabenazine has been previously reported in the FDA approved drug label AUSTEDOR indicated for the treatment of chorea associated with Huntington's disease and tardive dyskinesia in adults. Deutetrabenazine is administered twice daily (BID) up to a maximum daily dose of 48 mg.

Upon oral administration, deutetrabenazine is reduced to form four discrete isomeric secondary alcohol metabolites, collectively referred to as dihydrotetrabenazine (DHTBZ), which contains three asymmetric carbon centers (C-2, C-3, and C-11β), which could hypothetically result in eight stereoisomers. However, because the C-3 and C-11β carbons have fixed relative configurations, only four stereoisomers are possible: (R,R,R-DHTBZ or [+]-α-DHTBZ (alternate nomenclature); S,S,S-DHTBZ or [−]-α-DHTBZ; S,R,R-DHTBZ or [+]-β-DHTBZ; and R,S,S-DHTBZ or [−]-β-DHTBZ, which have the following structures:

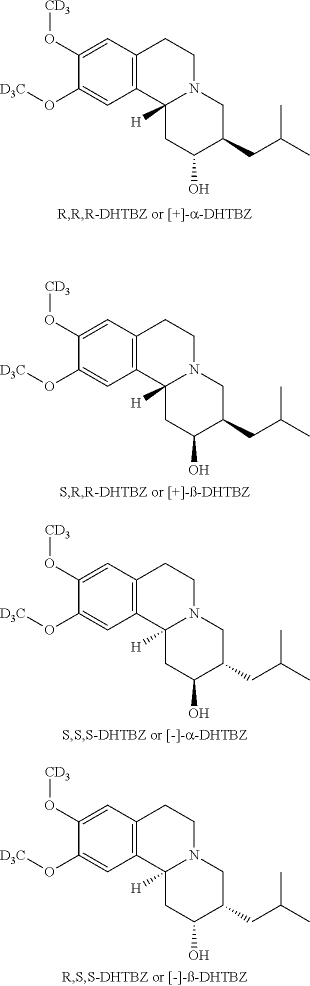 [SMILES: [2H]C([2H])([2H])Oc1cc2c(cc1OC([2H])([2H])[2H])[C@H]1C[C@@H](O)[C@H](CC(C)C)CN1CC2 ; [2H]C([2H])([2H])Oc1cc2c(cc1OC([2H])([2H])[2H])[C@H]1C[C@H](O)[C@H](CC(C)C)CN1CC2 ; [2H]C([2H])([2H])Oc1cc2c(cc1OC([2H])([2H])[2H])[C@@H]1C[C@H](O)[C@@H](CC(C)C)CN1CC2 ; [2H]C([2H])([2H])Oc1cc2c(cc1OC([2H])([2H])[2H])[C@@H]1C[C@@H](O)[C@@H](CC(C)C)CN1CC2]

The FDA approved drug label for AUSTEDOR indicates that although the pharmacokinetics of deutetrabenazine and its metabolites have not been systematically evaluated in patients who do not express the drug metabolizing enzyme, it is likely that the exposure to α-DHTBZ and β-DHTBZ would be increased similarly to taking a strong CYP2D6 inhibitor (approximately 3-fold). In patients who are CYP2D6 poor metabolizers, the daily dose of AUSTEDOR should not exceed 36 mg (maximum single dose of 18 mg).

The relative contribution of the four individual DHTBZ isomers to the clinical activity of deutetrabenazine has not been reported; metabolite data has been reported as the sum of all isomers or the sum of α and β isomers. There is a significant, unmet need for methods for administering a VMAT2 inhibitor, such as deutetrabenazine, to a patient in need thereof, wherein the patient is a CYP2D6 poor metabolizer. The present disclosure fulfills these and other needs, as evident in reference to the following disclosure.

### BRIEF SUMMARY

Provided is a method of treating a neurological or psychiatric disease or disorder in a patient in need thereof, comprising:
administering a maximum total daily dose of between about 6 mg/day and about 30 mg/day of a vesicular monoamine transporter 2 (VMAT2) inhibitor to the patient in need thereof, wherein the patient is a cytochrome P450 2D6 (CYP2D6) poor metabolizer, wherein the VMAT2 inhibitor is (RR,SS)-1, 3, 4, 6, 7, 11b-hexahydro-9, 10-di(methoxy-d3)-3-(2-methylpropyl)-2H-benzo[a]quinolizin-2-one (deutetrabenazine) or an enantiomer or pharmaceutically acceptable salt thereof.

Also provided is a method of treating a neurological or psychiatric disease or disorder in a patient in need thereof, comprising:
administering a maximum total daily dose of between about 6 mg/day and about 30 mg/day of a vesicular monoamine transporter 2 (VMAT2) inhibitor to the patient in need thereof, wherein the patient is a cytochrome P450 2D6 (CYP2D6) poor metabolizer,
wherein the VMAT2 inhibitor is deutetrabenazine or an enantiomer or pharmaceutically acceptable salt thereof.

Also provided is a method of treating a neurological or psychiatric disease or disorder in a patient in need thereof, comprising: administering to the patient a maximum daily total dose of deutetrabenazine or an enantiomer or pharmaceutically acceptable salt thereof, that is between about 6 mg/day and about 30 mg/day if, and because, the patient is a cytochrome P450 2D6 (CYP2D6) poor metabolizer.

Also provided is a method of treating a neurological or psychiatric disease or disorder in a patient in need thereof, wherein the patient is a cytochrome P450 2D6 (CYP2D6) poor metabolizer comprising:
administering to the patient a maximum total daily dose of between about 6 mg/day and about 30 mg/day of a vesicular monoamine transporter 2 (VMAT2) inhibitor,
wherein the VMAT2 inhibitor is deutetrabenazine or an enantiomer or pharmaceutically acceptable salt thereof,
monitoring the concentration of [−]-α-DHTBZ and/or [−]-β-DHTBZ in the patient's blood; and
reducing the amount of the VMAT2 inhibitor being administered when the [−]-α-DHTBZ and/or [−]-β-DHTBZ exposure in the patient's blood is increased as compared with the [−]-α-DHTBZ and/or [−]-β-DHTBZ level in a patient who is administered the VMAT2 inhibitor alone.

Also provided is a method of treating a neurological or psychiatric disease or disorder in a patient in need thereof, comprising:
administering a maximum total daily dose of 48 mg/day of a vesicular monoamine transporter 2 (VMAT2) inhibitor to the patient in need thereof, wherein the VMAT2 inhibitor is deutetrabenazine or an enantiomer or pharmaceutically acceptable salt thereof,
subsequently determining that the patient is a cytochrome P450 2D6 (CYP2D6) poor metabolizer, and
administering a maximum total daily dose of between 6 mg/day and about 30 mg/day of the vesicular monoamine transporter 2 (VMAT2) inhibitor to the patient, wherein the VMAT2 inhibitor is (RR,SS)-1, 3, 4, 6, 7, 11b-hexahydro-9, 10-di(methoxy-d3)-3-(2-methylpropyl)-2H-benzo[a]quinolizin-2-one (deutetrabenazine) or an enantiomer or pharmaceutically acceptable salt thereof.

Also provided is a method of decreasing a risk of QT prolongation in a patient being treated for a neurological or psychiatric disease or disorder with deutetrabenazine or an enantiomer or pharmaceutically acceptable salt thereof, comprising: administering to the patient a maximum total daily dose of deutetrabenazine or an enantiomer or pharmaceutically acceptable salt thereof, that is between about 6 mg/day and about 30 mg/day if, and because, the patient is a cytochrome P450 2D6 (CYP2D6) poor metabolizer.

Also provided is a method of reducing off-target activity and increasing on-target activity in a patient being treated for a neurological or psychiatric disease or disorder with deutetrabenazine or an enantiomer or pharmaceutically acceptable salt thereof, comprising: administering to the patient a maximum total daily dose of deutetrabenazine or an enantiomer or pharmaceutically acceptable salt thereof, that is between about 6 mg/day and about 30 mg/day if, and because, the patient is a cytochrome P450 2D6 (CYP2D6) poor metabolizer.

Also provided is a method of treating a neurological or psychiatric disease or disorder in a patient in need thereof wherein the patient is a cytochrome P450 2D6 (CYP2D6) poor metabolizer, comprising:
administering to the patient in need thereof a vesicular monoamine transporter 2 (VMAT2) inhibitor via a titration scheme that comprises the up-titration of the VMAT2 inhibitor from an initial dose in increments not exceeding about 6 mg/day at about weekly intervals until a maintenance dose is administered, wherein the maintenance dose does not exceed a total daily dose of about 30 mg of the VMAT2 inhibitor,
wherein the VMAT2 inhibitor is deutetrabenazine, or an enantiomer or pharmaceutically acceptable salt thereof.

In some embodiments, the VMAT2 inhibitor is deutetrabenazine. In some embodiments, the VMAT2 inhibitor is a pharmaceutically acceptable salt of deutetrabenazine. In some embodiments, the VMAT2 inhibitor is an enantiomer of deutetrabenazine. In some embodiments, the VMAT2 inhibitor is a pharmaceutically acceptable salt of an enantiomer of deutetrabenazine.

These and other aspects of the invention will be apparent upon reference to the following detailed description. To this end, various references are set forth herein which describe in more detail certain background information, procedures, compounds, and/or compositions, and are each hereby incorporated by reference in their entirety.

### BRIEF DESCRIPTION OF THE DRAWINGS

FIG. 1 depicts mean plasma concentration vs. time for each of the four 4 individual DHTBZ isomers.

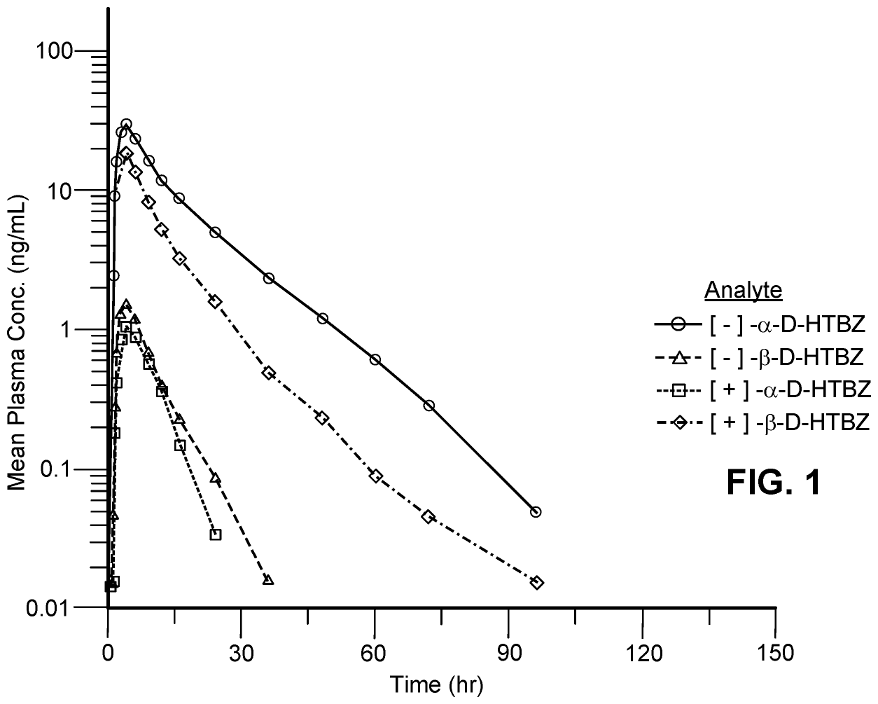

FIG. 2 depicts mean plasma concentration vs. time for the primary active metabolite from deutetrabenazine [+]-β-DHTBZ, for [+]-α-DHTBZ, and for the active metabolite of valbenazine, [+]-α-HTBZ.

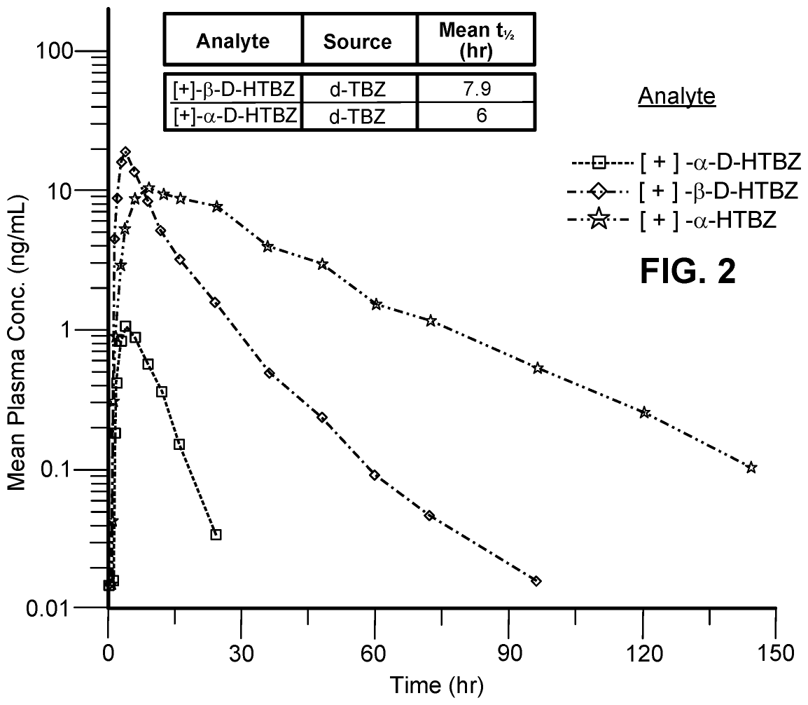

### DETAILED DESCRIPTION

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” or “some embodiments” or “a certain embodiment” means that a particular feature, structure or characteristic described in connection with the embodiment is included in at least one embodiment. Thus, the appearances of the phrases “in one embodiment” or “in an embodiment” or “in some embodiments” or “in a certain embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise.

As used herein, a substance is a “substrate” of enzyme activity when it can be chemically transformed by action of the enzyme on the substance. Substrates can be either activated or deactivated by the enzyme.

Cytochrome P450 2D6 (CYP2D6), a member of the cytochrome P450 mixed-function oxidase system, is one of the most important enzymes involved in the metabolism of xenobiotics in the body. The CYP2D6 gene is highly polymorphic, with more than 70 allelic variants described. The CYP2D6 function in any particular subject may be described as one of the following: poor metabolizers which are subjects having little or no CYP2D6 function; intermediate metabolizers which are subjects that metabolize drugs at a rate somewhere between the poor and extensive metabolizers; extensive metabolizers which are subjects having normal CYP2D6 function; and ultrarapid metabolizers which are subjects having multiple copies of the CYP2D6 gene expressed, and therefore greater-than-normal CYP2D6 function. Additionally, several cytochrome p450 isozymes are known to be genetically polymorphic, leading to altered substrate metabolizing ability in some individuals. Allelic variants of CYP2D6 are the best characterized, with many resulting in an enzyme with reduced, or no, catalytic activity. Gene duplication also occurs. As a result, four phenotypic subpopulations of metabolizers of CYP2D6 substrates exist: poor (PM), intermediate (IM), extensive (EM), and ultrarapid (UM).

As used herein, “the CYP2D6G1846A genotype” (also known as the CYP2D6*4 alleles, encompassing *4A, *4B, *4C, *4D, *4E, *4F, *4G, *4H, *4J, *4K, and *4L) means the polymorphism corresponding to nucleotide 3465 in GenBank sequence M33388.1 (GI:181303). CYP2D6G1846A polymorphism represents a G to A transition at the junction between intron 3 and exon 4, shifting the splice junction by one base pair, resulting in frameshift and premature termination of the protein.

As used herein, “the CYP2D6C100T genotype” (also known as the CYP2D6*10 and CYP2D6*14 alleles) means the polymorphism corresponding to nucleotide 1719 in GenBank sequence M33388.1 (GI:181303) or to nucleotide 100 in GenBank mRNA sequence M20403.1 (GI:181349). It is also referred to as the “CYP2D6P34S genotype”. The CYP2D6P34S/CYP2D6C100T polymorphism represents a C to T change that results in the substitution of a proline at position 34 by serine.

“Enzyme activity” refers broadly to the specific activity of the enzyme (i.e., the rate at which the enzyme transforms a substrate per mg or mole of enzyme) as well as the metabolic effect of such transformations.

A substance is an “inhibitor” of enzyme activity when the specific activity or the metabolic effect of the specific activity of the enzyme can be decreased by the presence of the substance, without reference to the precise mechanism of such decrease. For example, a substance can be an inhibitor of enzyme activity by competitive, non-competitive, allosteric or other type of enzyme inhibition, by decreasing expression of the enzyme, or other direct or indirect mechanisms. Co-administration of a given drug with an inhibitor may decrease the rate of metabolism of that drug through the metabolic pathway listed.

A substance is an “inducer” of enzyme activity when the specific activity or the metabolic effect of the specific activity of the enzyme can be increased by the presence of the substance, without reference to the precise mechanism of such increase. For example, a substance can be an inducer of enzyme activity by increasing reaction rate, by increasing expression of the enzyme, by allosteric activation or other direct or indirect mechanisms. Co-administration of a given drug with an enzyme inducer may increase the rate of excretion of the drug metabolized through the pathway indicated.

Any of these effects on enzyme activity can occur at a given concentration of active agent in a single sample, donor, or patient without regard to clinical significance. It is possible for a substance to be a substrate, inhibitor, or inducer of an enzyme activity. For example, the substance can be an inhibitor of enzyme activity by one mechanism and an inducer of enzyme activity by another mechanism. The function (substrate, inhibitor, or inducer) of the substance with respect to activity of an enzyme can depend on environmental conditions.

As used herein, a “strong CYP2D6 inhibitor” is a compound that increases the area under the concentration time curve (AUC) of a sensitive index substrate of the CYP2D6 pathway by >5-fold. Index substrates predictably exhibit exposure increase due to inhibition or induction of a given metabolic pathway and are commonly used in prospective clinical drug-drug interaction studies. Sensitive index substrates are index substrates that demonstrate an increase in AUC of ≥5-fold with strong index inhibitors of a given metabolic pathway in clinical drug-drug interaction studies. Examples of sensitive index substrates for the CYP2D6 pathway are fluoxetine and paroxetine. See, e.g., Drug Development and Drug Interactions: Table of Substrates, Inhibitor and Inducers at https://www.fda.gov/drugs/developmentapprovalprocess/developmentresources/druginteractionslabeling/ucm093664.htm and http://www.ildcare.eu/downloads/artseninfo/drugs_metabolized_by_cyp450s.pdf.

As used herein, in some embodiments, “pharmaceutically acceptable salt” refers to acid addition salts of deutetrabenazine with an inorganic or an organic acid. Lists of suitable salts are found in WO 87/05297, Johnston et al., published Sep. 11, 1987; Remington's Pharmaceutical Sciences, 17th ed., Mack Publishing Company, Easton, Pa., 1985, p. 1418; and J. Pharm. Sci., 66, 2 (1977), each of which is incorporated herein by reference in its entirety. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002 which is incorporated herein by reference in its entirety. The organic or inorganic acids include, but are not limited to, hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid, and the like. In some embodiments, “pharmaceutically acceptable salt” refers to base addition salts of deutetrabenazine with an inorganic or an organic base. Inorganic bases which may be used to prepare salts include, for example, sodium, potassium, lithium, ammonium, calcium, magnesium, iron, zinc, manganese, aluminum hydroxides, carbonates, bicarbonates, phosphates, and the like; particularly preferred are the ammonium, potassium, sodium, calcium, and magnesium hydroxides, carbonates, bicarbonates, or phosphates. Organic bases from which may be used to prepare salts include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

As used herein, “hyperkinetic disorder” or “hyperkinetic movement disorder” or “hyperkinesias” refers to disorders or diseases characterized by excessive, abnormal, involuntary movements. These neurological disorders include tremor, dystonia, myoclonus, athetosis, Huntington's disease, tardive dyskinesia, Tourette syndrome, dystonia, hemiballismus, chorea, senile chorea, or tics.

As used herein, “tardive syndrome” encompasses but is not limited to tardive dyskinesia, tardive dystonia, tardive akathisia, tardive tics, myoclonus, tremor and withdrawal-emergent syndrome. Tardive dyskinesia is characterized by rapid, repetitive, stereotypic, involuntary movements of the face, limbs, or trunk.

As used herein, “about” means±20% of the stated value, and includes more specifically values of ±10%, ±5%, ±2% and ±1% of the stated value.

As used herein, “AUC” refers to the area under the curve, or the integral, of the plasma concentration of an active pharmaceutical ingredient or metabolite over time following a dosing event.

As used herein “AUC₀₋ₜ” is the integral under the plasma concentration curve from time 0 (dosing) to time “t”.

As used herein, “AUC_{0-∞}” or AUC_{inf}” is the AUC from time 0 (dosing) to time infinity. Unless otherwise stated, AUC refers to AUC_{0-∞}.

As used herein, Cₘₐₓ is a pharmacokinetic parameter denoting the maximum observed blood plasma concentration following delivery of an active pharmaceutical ingredient. Cₘₐₓ occurs at the time of maximum plasma concentration, tₘₐₓ.

As used herein, “co-administer” and “co-administration” and variants thereof mean the administration of at least two drugs to a patient either subsequently, simultaneously, or consequently proximate in time to one another (e.g., within the same day, or week or period of 30 days, or sufficiently proximate that each of the at least two drugs can be simultaneously detected in the blood plasma). When co-administered, two or more active agents can be co-formulated as part of the same composition or administered as separate formulations. This also may be referred to herein as “concomitant” administration or variants thereof.

As used herein, “adjusting administration”, “altering administration”, “adjusting dosing”, or “altering dosing” are all equivalent and mean tapering off, reducing or increasing the dose of the substance, ceasing to administer the substance to the patient, or substituting a different active agent for the substance.

As used herein, “administering to a patient” refers to the process of introducing a composition or dosage form into the patient via an art-recognized means of introduction.

As used herein the term “disorder” is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

As used herein, a “dose” means the measured quantity of an active agent to be taken at one time by a patient. In certain embodiments, wherein the active agent is not deutetrabenazine free base, the quantity is the molar equivalent to the corresponding amount of deutetrabenazine free base. For example, often a drug is packaged in a pharmaceutically acceptable salt form, and the dosage for strength refers to the mass of the molar equivalent of the corresponding free base. For example, 18 mg/day used herein refers to 18 mg of deutetrabenazine free base per day.

As used herein, a “dosage” is the prescribed administration of a specific amount, number, and frequency of doses over a specific period of time.

As used herein, “effective amount” and “therapeutically effective amount” of an agent, compound, drug, composition or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician.

As used herein, “informing” means referring to or providing published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

As used herein, “labeling” means all labels or other means of written, printed, graphic, electronic, verbal, or demonstrative communication that is upon a pharmaceutical product or a dosage form or accompanying such pharmaceutical product or dosage form.

As used herein, “a medical care worker” means a worker in the health care field who may need or utilize information regarding an active agent, including a dosage form thereof, including information on safety, efficacy, dosing, administration, or pharmacokinetics. Examples of medical care workers include physicians, pharmacists, physician's assistants, nurses, aides, caretakers (which can include family members or guardians), emergency medical workers, and veterinarians.

As used herein, “Medication Guide” means an FDA-approved patient labeling for a pharmaceutical product conforming to the specifications set forth in 21 CFR 208 and other applicable regulations which contains information for patients on how to safely use a pharmaceutical product. A medication guide is scientifically accurate and is based on, and does not conflict with, the approved professional labeling for the pharmaceutical product under 21 CFR 201.57, but the language need not be identical to the sections of approved labeling to which it corresponds. A medication guide is typically available for a pharmaceutical product with special risk management information.

As used herein, “patient” or “individual” or “subject” means a mammal, including a human, for whom or which therapy is desired, and generally refers to the recipient of the therapy.

As used herein, “patient package insert” means information for patients on how to safely use a pharmaceutical product that is part of the FDA-approved labeling. It is an extension of the professional labeling for a pharmaceutical product that may be distributed to a patient when the product is dispensed which provides consumer-oriented information about the product in lay language, for example it may describe benefits, risks, how to recognize risks, dosage, or administration.

As used herein, “pharmaceutically acceptable” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

As used herein, a “product” or “pharmaceutical product” means a dosage form of an active agent plus published material, and optionally packaging.

As used herein, “product insert” means the professional labeling (prescribing information) for a pharmaceutical product, a patient package insert for the pharmaceutical product, or a medication guide for the pharmaceutical product.

As used herein, “professional labeling” or “prescribing information” means the official description of a pharmaceutical product approved by a regulatory agency (e.g., FDA or EMEA) regulating marketing of the pharmaceutical product, which includes a summary of the essential scientific information needed for the safe and effective use of the drug, such as, for example indication and usage; dosage and administration; who should take it; adverse events (side effects); instructions for use in special populations (pregnant women, children, geriatric, etc.); safety information for the patient, and the like.

As used herein, “published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, an audio recording, or other recording or electronic medium.

As used herein, “risk” means the probability or chance of adverse reaction, injury, or other undesirable outcome arising from a medical treatment. An “acceptable risk” means a measure of the risk of harm, injury, or disease arising from a medical treatment that will be tolerated by an individual or group. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small, or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At risk” means in a state or condition marked by a high level of risk or susceptibility. Risk assessment consists of identifying and characterizing the nature, frequency, and severity of the risks associated with the use of a product.

As used herein, “safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

As used herein, “tₘₐₓ” is a pharmacokinetic parameter denoting the time to maximum blood plasma concentration following delivery of an active pharmaceutical ingredient

As used herein, “t_{1/2}” or “plasma half-life” or “elimination half-life” or the like is a pharmacokinetic parameter denoting the apparent plasma terminal phase half-life, i.e., the time, after absorption and distribution of a drug is complete, for the plasma concentration to fall by half.

As used herein, “treating” or “treatment” refers to therapeutic applications to slow or stop progression of a disorder, prophylactic application to prevent development of a disorder, and/or reversal of a disorder. Reversal of a disorder differs from a therapeutic application which slows or stops a disorder in that with a method of reversing, not only is progression of a disorder completely stopped, cellular behavior is moved to some degree, toward a normal state that would be observed in the absence of the disorder.

As used herein, “up-titration” of a compound refers to increasing the amount of a compound to achieve a therapeutic effect that occurs before dose-limiting intolerability for the patient. Up-titration can be achieved in one or more dose increments, which may be the same or different.

As used herein, “VMAT2” refers to human vesicular monoamine transporter isoform 2, an integral membrane protein that acts to transport monoamines, particularly neurotransmitters such as dopamine, norepinephrine, serotonin, and histamine, from cellular cytosol into synaptic vesicles.

As used herein, the term “VMAT2 inhibitor”, “inhibit VMAT2”, or “inhibition of VMAT2” refers to the ability of a compound disclosed herein to alter the function of VMAT2. A VMAT2 inhibitor may block or reduce the activity of VMAT2 by forming a reversible or irreversible covalent bond between the inhibitor and VMAT2 or through formation of a noncovalently bound complex. Such inhibition may be manifest only in particular cell types or may be contingent on a particular biological event. The term “VMAT2 inhibitor”, “inhibit VMAT2”, or “inhibition of VMAT2” also refers to altering the function of VMAT2 by decreasing the probability that a complex forms between a VMAT2 and a natural substrate.

As used herein, “valbenazine” may be referred to as (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester; or as L-Valine, (2R,3R,11bR)-1,3,4,6,7,11b-hexahydro-9,10-dimethoxy-3-(2-methylpropyl)-2H-benzo[a]quinolizin-2-yl ester or as NBI-98854 and has the following chemical structure:

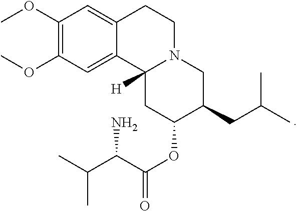 [SMILES: COc1cc2c(cc1OC)[C@H]1C[C@@H](OC(=O)[C@@H](N)C(C)C)[C@H](CC(C)C)CN1CC2]

A formulation of valbenazine:4-toluenesulfonate (1:2) (referred to herein as “valbenazine ditosylate”) has been previously reported in the FDA approved drug label INGREZZA® which is indicated for the treatment of adults with tardive dyskinesia.

As used herein, “[+]-α-HTBZ” means the compound which is an active metabolite of valbenazine having the structure:

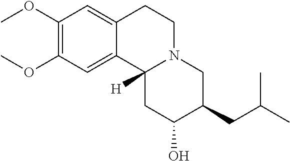 [SMILES: COc1cc2c(cc1OC)[C@H]1C[C@@H](O)[C@H](CC(C)C)CN1CC2]

[+]-α-HTBZ may be referred to as (2R,3R, 11bR) or as R,R,R-HTBZ or as (+)-α-3-isobutyl-9,10-dimethoxy-1,3,4,6,7,11b-hexahydro-2H-pyrido[2,1-a]isoquinolin-2-ol; or as (2R,3R,11bR)-3-isobutyl-9,10-dimethoxy-1,3,4,6,7,11b-hexahydro-2H-pyrido[2,1-a]isoquinolin-2-ol or as NBI-98782.

As used herein, “maximum total daily dose” or “maximum recommended total daily dose” or “maximum recommended daily dosage” or “maximum total daily dose” or “maximum daily dosage” or “total daily dosage” refers to the highest safe dosage of drug to be administered on a daily basis following dosage titration, i.e., the maintenance dose, as determined by a titration scheme, should not exceed the maximum recommended total daily dose. For example, the prescribing information for AUSTEDO revised December 2020 indicates that post-titration, a maximum recommended daily dosage of deutetrabenazine is 48 mg (24 mg twice daily) in patients who are not receiving CYP2D6 poor metabolizers and a total daily dosage of 36 mg per day (18 mg twice daily) for patients who is a CYP2D6 poor metabolizer.

Provided is a method of treating a neurological or psychiatric disease or disorder in a patient in need thereof, comprising:
administering a maximum total daily dose of between about 6 mg/day and about 30 mg/day of a vesicular monoamine transporter 2 (VMAT2) inhibitor to the patient in need thereof, wherein the patient is a cytochrome P450 2D6 (CYP2D6) poor metabolizer,
wherein the VMAT2 inhibitor is (RR,SS)-1, 3, 4, 6, 7, 11b-hexahydro-9, 10-di(methoxy-d3)-3-(2-methylpropyl)-2H-benzo[a]quinolizin-2-one (deutetrabenazine) or an enantiomer or pharmaceutically acceptable salt thereof.

Also provided is a method of treating a neurological or psychiatric disease or disorder in a patient in need thereof, comprising:
administering a maximum total daily dose of between about 6 mg/day and about 30 mg/day of a vesicular monoamine transporter 2 (VMAT2) inhibitor to the patient in need thereof, wherein the patient is a cytochrome P450 2D6 (CYP2D6) poor metabolizer,
wherein the VMAT2 inhibitor is deutetrabenazine or an enantiomer or pharmaceutically acceptable salt thereof.

Also provided is a method of treating a neurological or psychiatric disease or disorder in a patient in need thereof, comprising:
administering to the patient a maximum total daily dose of deutetrabenazine or an enantiomer or pharmaceutically acceptable salt thereof, that is between about 6 mg/day and about 30 mg/day if, and because, the patient is a cytochrome P450 2D6 (CYP2D6) poor metabolizer.

Also provided is a method of treating a neurological or psychiatric disease or disorder in a patient in need thereof, wherein the patient is a cytochrome P450 2D6 (CYP2D6) poor metabolizer comprising:
administering to the patient a maximum total daily dose of between about 6 mg/day and about 30 mg/day of a vesicular monoamine transporter 2 (VMAT2) inhibitor,
wherein the VMAT2 inhibitor is deutetrabenazine or an enantiomer or pharmaceutically acceptable salt thereof,
monitoring the concentration of [−]-α-DHTBZ and/or [−]-β-DHTBZ in the patient's blood; and
reducing the amount of the VMAT2 inhibitor being administered when the [−]-α-DHTBZ and/or [−]-β-DHTBZ exposure in the patient's blood is increased as compared with the [−]-α-DHTBZ and/or [−]-β-DHTBZ level in a patient who is not a CYPD2D6 poor metabolizer.

In some embodiments, the [−]-α-DHTBZ and [−]-β-DHTBZ exposures are measured as the area under the plasma concentration versus time curve from 0 hours extrapolated to infinity or measured as the maximum observed blood plasma concentration (Cₘₐₓ) at the time of maximum plasma concentration (tₘₐₓ).

In some embodiments, the [−]-α-DHTBZ and [−]-β-DHTBZ exposures are measured as the maximum observed blood plasma concentration (Cₘₐₓ).

In some embodiments, the increased [−]-α-DHTBZ and/or [−]-β-DHTBZ exposure increases the risk of one or more exposure-related adverse reactions.

Also provided is a method of treating a neurological or psychiatric disease or disorder in a patient in need thereof, comprising:
administering a maximum total daily dose of about 48 mg/day of a vesicular monoamine transporter 2 (VMAT2) inhibitor to the patient in need thereof, wherein the VMAT2 inhibitor is deutetrabenazine or an enantiomer or pharmaceutically acceptable salt thereof,
subsequently determining that the patient is a CYP2D6 poor metabolizer, and
administering a maximum total daily dose of between about 6 mg/day and about 30 mg/day of a vesicular monoamine transporter 2 (VMAT2) inhibitor to the patient in need thereof.

Also provided is a method of administering deutetrabenazine or an enantiomer or pharmaceutically acceptable salt thereof to a patient in need thereof, wherein the patient is a CYP2D6 poor metabolizer, comprising administering a total daily dosage of deutetrabenazine or an enantiomer or pharmaceutically acceptable salt thereof that does not exceed about 30 mg. In some embodiments, the total daily dose is administered in a maximum single dose of about 15 mg twice per day. In some embodiments, the maximum daily dose is administered once daily.

Also provided is a method of administering deutetrabenazine or an enantiomer or pharmaceutically acceptable salt thereof to a patient in need thereof, wherein the patient is a CYP2D6 poor metabolizer, comprising administering a total daily dosage of deutetrabenazine or an enantiomer or pharmaceutically acceptable salt thereof that does not exceed about 25 mg. In some embodiments, the total daily dose is administered in a maximum single dose of about 12.5 mg twice per day. In some embodiments, the maximum daily dose is administered once daily.

Also provided is a method of administering deutetrabenazine or an enantiomer or pharmaceutically acceptable salt thereof to a patient in need thereof, wherein the patient is a CYP2D6 poor metabolizer, comprising administering a total daily dosage of deutetrabenazine or an enantiomer or pharmaceutically acceptable salt thereof that does not exceed about 27 mg. In some embodiments, the total daily dose is administered in a maximum single dose of about 13.5 mg twice per day. In some embodiments, the maximum daily dose is administered once daily.

Also provided is a method of administering deutetrabenazine or an enantiomer or pharmaceutically acceptable salt thereof to a patient in need thereof, wherein the patient is a CYP2D6 poor metabolizer, comprising administering a total daily dosage of deutetrabenazine or an enantiomer or pharmaceutically acceptable salt thereof that does not exceed about 24 mg. In some embodiments, the total daily dose is administered in a maximum single dose of about 12 mg twice per day. In some embodiments, the maximum daily dose is administered once daily.

Also provided is a method of administering deutetrabenazine or an enantiomer or pharmaceutically acceptable salt thereof to a patient in need thereof, wherein the patient is a CYP2D6 poor metabolizer, comprising administering a total daily dosage of deutetrabenazine or an enantiomer or pharmaceutically acceptable salt thereof that does not exceed about 21 mg. In some embodiments, the total daily dose is administered in a maximum single dose of about 10.5 mg twice per day. In some embodiments, the maximum daily dose is administered once daily.

Also provided is a method of administering deutetrabenazine or an enantiomer or pharmaceutically acceptable salt thereof to a patient in need thereof, wherein the patient is a CYP2D6 poor metabolizer, comprising administering a total daily dosage of deutetrabenazine or an enantiomer or pharmaceutically acceptable salt thereof that does not exceed about 20 mg. In some embodiments, the total daily dose is administered in a maximum single dose of about 10 mg twice per day. In some embodiments, the maximum daily dose is administered once daily.

Also provided is a method of administering deutetrabenazine or an enantiomer or pharmaceutically acceptable salt thereof to a patient in need thereof, wherein the patient is a CYP2D6 poor metabolizer, comprising administering a total daily dosage of deutetrabenazine or an enantiomer or pharmaceutically acceptable salt thereof that does not exceed about 18 mg. In some embodiments, the total daily dose is administered in a maximum single dose of about 9 mg twice per day. In some embodiments, the maximum daily dose is administered once daily.

Also provided is a method of administering deutetrabenazine or an enantiomer or pharmaceutically acceptable salt thereof to a patient in need thereof, wherein the patient is a CYP2D6 poor metabolizer, comprising administering a total daily dosage of deutetrabenazine or an enantiomer or pharmaceutically acceptable salt thereof that does not exceed about 15 mg. In some embodiments, the total daily dose is administered in a maximum single dose of about 7.5 mg twice per day. In some embodiments, the maximum daily dose is administered once daily.

Also provided is a method of administering deutetrabenazine or an enantiomer or pharmaceutically acceptable salt thereof to a patient in need thereof, wherein the patient is a CYP2D6 poor metabolizer, comprising administering a total daily dosage of deutetrabenazine or an enantiomer or pharmaceutically acceptable salt thereof that does not exceed about 12 mg. In some embodiments, the total daily dose is administered in a maximum single dose of about 6 mg twice per day. In some embodiments, the maximum daily dose is administered once daily.

Also provided is a method of administering deutetrabenazine or an enantiomer or pharmaceutically acceptable salt thereof to a patient in need thereof, wherein the patient is a CYP2D6 poor metabolizer, comprising administering a total daily dosage of deutetrabenazine or an enantiomer or pharmaceutically acceptable salt thereof that does not exceed about 10 mg. In some embodiments, the total daily dose is administered in a maximum single dose of about 5 mg twice per day. In some embodiments, the maximum daily dose is administered once daily.

Also provided is a method of administering deutetrabenazine or an enantiomer or pharmaceutically acceptable salt thereof to a patient in need thereof, wherein the patient is a CYP2D6 poor metabolizer, comprising administering a total daily dosage of deutetrabenazine or an enantiomer or pharmaceutically acceptable salt thereof that does not exceed about 6 mg. In some embodiments, the total daily dose is administered in a maximum single dose of about 3 mg twice per day. In some embodiments, the maximum daily dose is administered once daily.

Also provided is a method of decreasing a risk of QT prolongation in a patient being treated for a neurological or psychiatric disease or disorder with deutetrabenazine or an enantiomer or pharmaceutically acceptable salt thereof, comprising:

administering to the patient a maximum total daily dose of deutetrabenazine or an enantiomer or pharmaceutically acceptable salt thereof, that is between about 6 mg/day and about 30 mg/day if, and because, the patient is a CYP2D6 poor metabolizer.

Also provided is a method of reducing off-target activity and increasing on-target activity in a patient being treated for a neurological or psychiatric disease or disorder with deutetrabenazine or an enantiomer or pharmaceutically acceptable salt thereof, comprising:

administering to the patient a maximum total daily dose of deutetrabenazine or an enantiomer or pharmaceutically acceptable salt thereof, that is between about 6 mg/day and about 30 mg/day if, and because, the is a CYP2D6 poor metabolizer.

In some embodiments, the off-target effects are caused by (−)-α-3-isobutyl-9,10-di(methoxy-d3)-1,3,4,6,7, 11b-hexahydro-2H-pyrido[2,1-a]isoquinolin-2-ol ([−]-α-DHTBZ) and (−)-β-3-isobutyl-9,10-di(methoxy-d3)-1,3,4,6,7,11b-hexahydro-2H-pyrido[2,1-a]isoquinolin-2-ol ([−]-β-DHTBZ).

In some embodiments, the [−]-α-DHTBZ and [−]-β-DHTBZ activate an off-target receptor chosen from serotonin 5-HT7 receptor and dopamine D2 receptors.

In some embodiments, the off-target effects are caused by [−]-α-DHTBZ and [−]-β-DHTBZ.

In some embodiments, the off-target effects are caused by [−]-α-DHTBZ.

In some embodiments, the off-target effects are caused by [−]-β-DHTBZ.

In some embodiments, the on-target effects are caused by (+)-β-3-isobutyl-9,10-di(methoxy-d3)-1,3,4,6,7,11b-hexahydro-2H-pyrido[2,1-a]isoquinolin-2-ol ([+]-β-DHTBZ).

Also provided is a method of reducing off-target activity and increasing on-target activity in a patient being treated for a neurological or psychiatric disease or disorder with deutetrabenazine, or an enantiomer or pharmaceutically acceptable salt thereof, comprising: administering to the patient a dose of deutetrabenazine, or an enantiomer or pharmaceutically acceptable salt thereof, to achieve a maximal blood plasma concentration (Cₘₐₓ) of [−]-α-DHTBZ of about 50 ng/mL.

Also provided is a method of treating chorea associated with Huntington's disease in a patient in need thereof, wherein the patient is a CYP2D6 poor metabolizer, comprising:
administering an initial dose of a vesicular monoamine transporter 2 (VMAT2) inhibitor to the patient in need thereof, wherein the VMAT2 inhibitor is deutetrabenazine or a pharmaceutically acceptable salt thereof;
carrying out an up-titration scheme at weekly intervals by about 6 mg/day of the VMAT2 inhibitor; and
monitoring the reduction of symptoms of chorea associated with Huntington's disease and tolerability of the patient to the VMAT2 inhibitor,
wherein the total daily dose of the VMAT2 inhibitor does not exceed 24 mg and any single dose not exceeding 12 mg/day.

In some embodiments, the VMAT2 inhibitor is deutetrabenazine.

In some embodiments, the initial dose is about 3 mg/day. In some embodiments, the initial dose is about 4 mg/day. In some embodiments, the initial dose is about 5 mg/day. In some embodiments, the initial dose is about 6 mg/day.

Also provided is a method of treating chorea associated with Huntington's disease in a patient in need thereof, wherein the patient is a CYP2D6 poor metabolizer, comprising:
administering an initial dose of a vesicular monoamine transporter 2 (VMAT2) inhibitor to the patient in need thereof, wherein the VMAT2 inhibitor is deutetrabenazine or a pharmaceutically acceptable salt thereof;
carrying out an up-titration scheme at weekly intervals by about 6 mg/day of the VMAT2 inhibitor; and
monitoring the reduction of symptoms of chorea associated with Huntington's disease and tolerability of the patient to the VMAT2 inhibitor,
wherein the total daily dose of the VMAT2 inhibitor does not exceed 18 mg and any single dose not exceeding 9 mg/day.

In some embodiments, the VMAT2 inhibitor is deutetrabenazine.

In some embodiments, the initial dose is about 3 mg/day. In some embodiments, the initial dose is about 4 mg/day. In some embodiments, the initial dose is about 5 mg/day. In some embodiments, the initial dose is about 6 mg/day.

Also provided is a method of treating chorea associated with Huntington's disease in a patient in need thereof, wherein the patient is a CYP2D6 poor metabolizer, comprising:
administering an initial dose of a vesicular monoamine transporter 2 (VMAT2) inhibitor to the patient in need thereof, wherein the VMAT2 inhibitor is deutetrabenazine or a pharmaceutically acceptable salt thereof;
carrying out an up-titration scheme at weekly intervals by about 6 mg/day of the VMAT2 inhibitor; and
monitoring the reduction of symptoms of chorea associated with Huntington's disease and tolerability of the patient to the VMAT2 inhibitor,
wherein the total daily dose of the VMAT2 inhibitor does not exceed 12 mg and any single dose not exceeding 6 mg/day.

In some embodiments, the VMAT2 inhibitor is deutetrabenazine.

In some embodiments, the initial dose is about 3 mg/day. In some embodiments, the initial dose is about 4 mg/day. In some embodiments, the initial dose is about 5 mg/day. In some embodiments, the initial dose is about 6 mg/day.

Also provided is a method of treating chorea associated with Huntington's disease in a patient in need thereof, wherein the patient is a CYP2D6 poor metabolizer, comprising:
administering an initial dose of a vesicular monoamine transporter 2 (VMAT2) inhibitor to the patient in need thereof, wherein the VMAT2 inhibitor is deutetrabenazine or a pharmaceutically acceptable salt thereof;
carrying out an up-titration scheme at weekly intervals by about 3 mg/day of the VMAT2 inhibitor; and
monitoring the reduction of symptoms of chorea associated with Huntington's disease and tolerability of the patient to the VMAT2 inhibitor,
wherein the total daily dose of the VMAT2 inhibitor does not exceed 24 mg and any single dose not exceeding 12 mg/day.

In some embodiments, the VMAT2 inhibitor is deutetrabenazine.

In some embodiments, the initial dose is about 3 mg/day. In some embodiments, the initial dose is about 4 mg/day. In some embodiments, the initial dose is about 5 mg/day. In some embodiments, the initial dose is about 6 mg/day.

Also provided is a method of treating chorea associated with Huntington's disease in a patient in need thereof, wherein the patient the patient is a CYP2D6 poor metabolizer, comprising:
administering an initial dose of a vesicular monoamine transporter 2 (VMAT2) inhibitor to the patient in need thereof, wherein the VMAT2 inhibitor is deutetrabenazine or a pharmaceutically acceptable salt thereof;
carrying out an up-titration scheme at weekly intervals by about 3 mg/day of the VMAT2 inhibitor; and
monitoring the reduction of symptoms of chorea associated with Huntington's disease and tolerability of the patient to the VMAT2 inhibitor,
wherein the total daily dose of the VMAT2 inhibitor does not exceed 18 mg and any single dose not exceeding 9 mg/day.

In some embodiments, the VMAT2 inhibitor is deutetrabenazine.

In some embodiments, the initial dose is about 3 mg/day. In some embodiments, the initial dose is about 4 mg/day. In some embodiments, the initial dose is about 5 mg/day. In some embodiments, the initial dose is about 6 mg/day.

Also provided is a method of treating chorea associated with Huntington's disease in a patient in need thereof, wherein the patient the patient is a CYP2D6 poor metabolizer, comprising:
administering an initial dose of a vesicular monoamine transporter 2 (VMAT2) inhibitor to the patient in need thereof, wherein the VMAT2 inhibitor is deutetrabenazine or a pharmaceutically acceptable salt thereof;
carrying out an up-titration scheme at weekly intervals by about 3 mg/day of the VMAT2 inhibitor; and
monitoring the reduction of symptoms of chorea associated with Huntington's disease and tolerability of the patient to the VMAT2 inhibitor,
wherein the total daily dose of the VMAT2 inhibitor does not exceed 12 mg and any single dose not exceeding 6 mg/day.

In some embodiments, the VMAT2 inhibitor is deutetrabenazine.

In some embodiments, the initial dose is about 3 mg/day. In some embodiments, the initial dose is about 4 mg/day. In some embodiments, the initial dose is about 5 mg/day. In some embodiments, the initial dose is about 6 mg/day.

Also provided is a method of treating tardive dyskinesia in a patient in need thereof, wherein the patient the patient is a CYP2D6 poor metabolizer, comprising:
administering an initial dose of a vesicular monoamine transporter 2 (VMAT2) inhibitor to the patient in need thereof, wherein the VMAT2 inhibitor is deutetrabenazine or a pharmaceutically acceptable salt thereof;
carrying out an up-titration scheme at weekly intervals by about 6 mg/day of the VMAT2 inhibitor; and
monitoring the reduction of symptoms of chorea associated with Huntington's disease and tolerability of the patient to the VMAT2 inhibitor,
wherein the total daily dose of the VMAT2 inhibitor does not exceed 24 mg and any single dose not exceeding 12 mg/day.

In some embodiments, the VMAT2 inhibitor is deutetrabenazine.

In some embodiments, the initial dose is about 9 mg/day. In some embodiments, the initial dose is about 10 mg/day. In some embodiments, the initial dose is about 11 mg/day. In some embodiments, the initial dose is about 12 mg/day.

Also provided is a method of treating tardive dyskinesia in a patient in need thereof, wherein the patient the patient is a CYP2D6 poor metabolizer, comprising:
administering an initial dose of a vesicular monoamine transporter 2 (VMAT2) inhibitor to the patient in need thereof, wherein the VMAT2 inhibitor is deutetrabenazine or a pharmaceutically acceptable salt thereof;
carrying out an up-titration scheme at weekly intervals by about 6 mg/day of the VMAT2 inhibitor; and
monitoring the reduction of symptoms of chorea associated with Huntington's disease and tolerability of the patient to the VMAT2 inhibitor,
wherein the total daily dose of the VMAT2 inhibitor does not exceed 18 mg and any single dose not exceeding 9 mg/day.

In some embodiments, the VMAT2 inhibitor is deutetrabenazine.

In some embodiments, the initial dose is about 9 mg/day. In some embodiments, the initial dose is about 10 mg/day. In some embodiments, the initial dose is about 11 mg/day. In some embodiments, the initial dose is about 12 mg/day.

Also provided is a method of treating tardive dyskinesia in a patient in need thereof, wherein the patient the patient is a CYP2D6 poor metabolizer, comprising:
administering an initial dose of a vesicular monoamine transporter 2 (VMAT2) inhibitor to the patient in need thereof, wherein the VMAT2 inhibitor is deutetrabenazine or a pharmaceutically acceptable salt thereof;
carrying out an up-titration scheme at weekly intervals by about 6 mg/day of the VMAT2 inhibitor; and
monitoring the reduction of symptoms of chorea associated with Huntington's disease and tolerability of the patient to the VMAT2 inhibitor,
wherein the total daily dose of the VMAT2 inhibitor does not exceed 12 mg and any single dose not exceeding 6 mg/day.

In some embodiments, the VMAT2 inhibitor is deutetrabenazine.

In some embodiments, the initial dose is about 9 mg/day. In some embodiments, the initial dose is about 10 mg/day. In some embodiments, the initial dose is about 11 mg/day. In some embodiments, the initial dose is about 12 mg/day.

Also provided is a method of treating tardive dyskinesia in a patient in need thereof, wherein the patient the patient is a CYP2D6 poor metabolizer, comprising:
administering an initial dose of a vesicular monoamine transporter 2 (VMAT2) inhibitor to the patient in need thereof, wherein the VMAT2 inhibitor is deutetrabenazine or a pharmaceutically acceptable salt thereof;
carrying out an up-titration scheme at weekly intervals by about 3 mg/day of the VMAT2 inhibitor; and
monitoring the reduction of symptoms of chorea associated with Huntington's disease and tolerability of the patient to the VMAT2 inhibitor,
wherein the total daily dose of the VMAT2 inhibitor does not exceed 24 mg and any single dose not exceeding 12 mg/day.

In some embodiments, the VMAT2 inhibitor is deutetrabenazine.

In some embodiments, the initial dose is about 9 mg/day. In some embodiments, the initial dose is about 10 mg/day. In some embodiments, the initial dose is about 11 mg/day. In some embodiments, the initial dose is about 12 mg/day.

Also provided is a method of treating tardive dyskinesia in a patient in need thereof, wherein the patient the patient is a CYP2D6 poor metabolizer, comprising:
administering an initial dose of a vesicular monoamine transporter 2 (VMAT2) inhibitor to the patient in need thereof, wherein the VMAT2 inhibitor is deutetrabenazine or a pharmaceutically acceptable salt thereof;
carrying out an up-titration scheme at weekly intervals by about 3 mg/day of the VMAT2 inhibitor; and
monitoring the reduction of symptoms of chorea associated with Huntington's disease and tolerability of the patient to the VMAT2 inhibitor,
wherein the total daily dose of the VMAT2 inhibitor does not exceed 18 mg and any single dose not exceeding 9 mg/day.

In some embodiments, the VMAT2 inhibitor is deutetrabenazine.

In some embodiments, the initial dose is about 9 mg/day. In some embodiments, the initial dose is about 10 mg/day. In some embodiments, the initial dose is about 11 mg/day. In some embodiments, the initial dose is about 12 mg/day.

Also provided is a method of treating tardive dyskinesia in a patient in need thereof, wherein the patient the patient is a CYP2D6 poor metabolizer, comprising:
administering an initial dose of a vesicular monoamine transporter 2 (VMAT2) inhibitor to the patient in need thereof, wherein the VMAT2 inhibitor is deutetrabenazine or a pharmaceutically acceptable salt thereof;
carrying out an up-titration scheme at weekly intervals by about 3 mg/day of the VMAT2 inhibitor; and
monitoring the reduction of symptoms of chorea associated with Huntington's disease and tolerability of the patient to the VMAT2 inhibitor,
wherein the total daily dose of the VMAT2 inhibitor does not exceed 12 mg and any single dose not exceeding 6 mg/day.

In some embodiments, the VMAT2 inhibitor is deutetrabenazine.

In some embodiments, the initial dose is about 9 mg/day. In some embodiments, the initial dose is about 10 mg/day. In some embodiments, the initial dose is about 11 mg/day. In some embodiments, the initial dose is about 12 mg/day.

Also provided is a method of reducing off-target activity and increasing on-target activity in a patient being treated for a neurological or psychiatric disease or disorder with deutetrabenazine, or an enantiomer or pharmaceutically acceptable salt thereof, comprising:
administering to the patient a dose of deutetrabenazine, or an enantiomer or pharmaceutically acceptable salt thereof, to achieve a maximal blood plasma concentration (Cₘₐₓ) of [−]-α-DHTBZ of about 50 ng/mL in a patient who is not a CYP2D6 poor metabolizer or of about 60 ng/mL when the patient is a CYP2D6 poor metabolizer.

In certain embodiments, the VMAT2 inhibitor is administered in an amount sufficient to achieve a maximal blood plasma concentration (Cₘₐₓ) of [−]-α-DHTBZ of about 47 ng/ml in a patient who is not a CYP2D6 poor metabolizer or of about 56 ng/ml when the patient is a CYP2D6 poor metabolizer.

In certain embodiments, the VMAT2 inhibitor is administered in an amount sufficient to achieve a maximal blood plasma concentration (Cₘₐₓ) of [−]-α-DHTBZ of about 35 ng/ml in a patient who is not a CYP2D6 poor metabolizer or of about 42 ng/ml in a patient who is a CYP2D6 poor metabolizer.

In certain embodiments, the VMAT2 inhibitor is administered in an amount sufficient to achieve a maximal blood plasma concentration (Cₘₐₓ) of [−]-α-DHTBZ of about 29 ng/ml in a patient who is not a CYP2D6 poor metabolizer or of about 34.8 ng/ml in a patient who is a CYP2D6 poor metabolizer.

In certain embodiments, the VMAT2 inhibitor is administered in an amount sufficient to achieve a maximal blood plasma concentration (Cₘₐₓ) of [−]-α-DHTBZ of about 23 ng/ml in a patient who is not a CYP2D6 poor metabolizer or of about 27.6 ng/ml in a patient who is a CYP2D6 poor metabolizer.

In certain embodiments, the VMAT2 inhibitor is administered in an amount sufficient to achieve a maximal blood plasma concentration (Cₘₐₓ) of [−]-α-DHTBZ of about 17.5 ng/ml in a patient who is not a CYP2D6 poor metabolizer or of about 21 ng/ml in a patient who is a CYP2D6 poor metabolizer.

In certain embodiments, the VMAT2 inhibitor is administered in an amount sufficient to achieve a maximal blood plasma concentration (Cₘₐₓ) of [−]-α-DHTBZ of about 11.7 ng/ml in a patient who is not a CYP2D6 poor metabolizer or of about 14 ng/ml in a patient who is a CYP2D6 poor metabolizer.

Also provided is a method of reducing off-target activity and increasing on-target activity in a patient being treated for a neurological or psychiatric disease or disorder with deutetrabenazine, or an enantiomer or pharmaceutically acceptable salt thereof, comprising:
administering to the patient a dose of deutetrabenazine, or an enantiomer or pharmaceutically acceptable salt thereof, to achieve a maximal AUC₍₀₋ₜₐᵤ₎ of [−]-α-DHTBZ of about 450 hr*ng/mL in a patient who is not a CYP2D6 poor metabolizer or of about 855 hr*ng/ml in a patient who is a CYP2D6 poor metabolizer.

In certain embodiments, the VMAT2 inhibitor is administered in an amount sufficient to achieve a maximal AUC₍₀₋ₜₐᵤ₎ of [−]-α-DHTBZ of about 397 hr*ng/ml in a patient who is not a CYP2D6 poor metabolizer or of about 754 hr*ng/ml in a patient who is a CYP2D6 poor metabolizer.

In certain embodiments, the VMAT2 inhibitor is administered in an amount sufficient to achieve a maximal AUC₍₀₋ₜₐᵤ₎ of [−]-α-DHTBZ of about 298 hr*ng/ml in a patient who is not a CYP2D6 poor metabolizer or of about 566 hr*ng/ml in a patient who is a CYP2D6 poor metabolizer.

In certain embodiments, the VMAT2 inhibitor is administered in an amount sufficient to achieve a maximal AUC₍₀₋ₜₐᵤ₎ of [−]-α-DHTBZ of about 248 hr*ng/ml in in a patient who is not a CYP2D6 poor metabolizer or of about 471 hr*ng/ml in a patient who is a CYP2D6 poor metabolizer.

In certain embodiments, the VMAT2 inhibitor is administered in an amount sufficient to achieve a maximal AUC₍₀₋ₜₐᵤ₎ of [−]-α-DHTBZ of about 199 hr*ng/ml in a patient who is not a CYP2D6 poor metabolizer or of about 378 hr*ng/ml in a patient who is a CYP2D6 poor metabolizer.

In certain embodiments, the VMAT2 inhibitor is administered in an amount sufficient to achieve a maximal AUC₍₀₋ₜₐᵤ₎ of [−]-α-DHTBZ of about 149 hr*ng/ml in a patient who is not a CYP2D6 poor metabolizer or of about 283 hr*ng/mL in a patient who is a CYP2D6 poor metabolizer.

In certain embodiments, the VMAT2 inhibitor is administered in an amount sufficient to achieve a maximal AUC₍₀₋ₜₐᵤ₎ of [−]-α-DHTBZ of about 99 hr*ng/mL in a patient who is not a CYP2D6 poor metabolizer or of about 188 hr*ng/mL in a patient who is a CYP2D6 poor metabolizer.

Also provided is a method of reducing off-target activity and increasing on-target activity in a patient being treated for a neurological or psychiatric disease or disorder with deutetrabenazine, or an enantiomer or pharmaceutically acceptable salt thereof, comprising:

administering to the patient a dose of deutetrabenazine or an enantiomer or pharmaceutically acceptable salt thereof, to achieve a maximal blood plasma concentration (Cₘₐₓ) of [−]-β-DHTBZ of about 2 ng/mL in a patient who is not a CYP2D6 poor metabolizer or of about 4.4 ng/ml in a patient who is a CYP2D6 poor metabolizer.

In certain embodiments, the VMAT2 inhibitor is administered in an amount sufficient to achieve a maximal blood plasma concentration (Cₘₐₓ) of [−]-β-DHTBZ of about 1.9 ng/mL in a patient who is not a CYP2D6 poor metabolizer or of about 4.2 ng/ml in a patient who is a CYP2D6 poor metabolizer.

In certain embodiments, the VMAT2 inhibitor is administered in an amount sufficient to achieve a maximal blood plasma concentration (Cₘₐₓ) of [−]-β-DHTBZ of about 1.4 ng/ml in a patient who is not a CYP2D6 poor metabolizer or of about 3.1 ng/mL in a patient who is a CYP2D6 poor metabolizer.

In certain embodiments, the VMAT2 inhibitor is administered in an amount sufficient to achieve a maximal blood plasma concentration (Cₘₐₓ) of [−]-β-DHTBZ of about 1.2 ng/ml in a patient who is not a CYP2D6 poor metabolizer or of about 2.6 ng/ml in a patient who is a CYP2D6 poor metabolizer.

In certain embodiments, the VMAT2 inhibitor is administered in an amount sufficient to achieve a maximal blood plasma concentration (Cₘₐₓ) of [−]-β-DHTBZ of about 0.9 ng/mL in a patient who is not a CYP2D6 poor metabolizer or of about 2.0 ng/ml in a patient who is a CYP2D6 poor metabolizer.

In certain embodiments, the VMAT2 inhibitor is administered in an amount sufficient to achieve a maximal blood plasma concentration (Cₘₐₓ) of [−]-β-DHTBZ of about 0.7 ng/mL in a patient who is not a CYP2D6 poor metabolizer or of about 1.5 ng/mL in a patient who is a CYP2D6 poor metabolizer.

In certain embodiments, the VMAT2 inhibitor is administered in an amount sufficient to achieve a maximal blood plasma concentration (Cₘₐₓ) of [−]-β-DHTBZ of about 0.5 ng/mL in a patient who is not a CYP2D6 poor metabolizer or of about 1.1 ng/mL in a patient who is a CYP2D6 poor metabolizer.

Also provided is a method of reducing off-target activity and increasing on-target activity in a patient being treated for a neurological or psychiatric disease or disorder with deutetrabenazine, or an enantiomer or pharmaceutically acceptable salt thereof, comprising:
administering to the patient a dose of deutetrabenazine, or an enantiomer or pharmaceutically acceptable salt thereof, to achieve a maximal AUC₍₀₋ₜₐᵤ₎ of [−]-β-DHTBZ of about 15 hr*ng/ml in a patient who is not a CYP2D6 poor metabolizer or of about 97.5 hr*ng/mL in a patient who is a CYP2D6 poor metabolizer.

In certain embodiments, the VMAT2 inhibitor is administered in an amount sufficient to achieve a maximal a maximal AUC₍₀₋ₜₐᵤ₎ of [−]-β-DHTBZ of about 13.5 hr*ng/mL in a patient who is not a CYP2D6 poor metabolizer or of about 88 hr*ng/ml in a patient who is a CYP2D6 poor metabolizer.

In certain embodiments, the VMAT2 inhibitor is administered in an amount sufficient to achieve a maximal AUC₍₀₋ₜₐᵤ₎ of [−]-β-DHTBZ of about 10 hr*ng/mL in a patient who is not a CYP2D6 poor metabolizer or of about 65 hr*ng/ml in a patient who is a CYP2D6 poor metabolizer.

In certain embodiments, the VMAT2 inhibitor is administered in an amount sufficient to achieve a maximal AUC₍₀₋ₜₐᵤ₎ of [−]-β-DHTBZ of about 8.4 hr*ng/ml in a patient who is not a CYP2D6 poor metabolizer or of about 54.6 hr*ng/ml in a patient who is a CYP2D6 poor metabolizer.

In certain embodiments, the VMAT2 inhibitor is administered in an amount sufficient to achieve a maximal AUC₍₀₋ₜₐᵤ₎ of [−]-β-DHTBZ of about 6.8 hr*ng/ml in a patient who is not a CYP2D6 poor metabolizer or of about 44 hr*ng/ml in a patient who is a CYP2D6 poor metabolizer.

In certain embodiments, the VMAT2 inhibitor is administered in an amount sufficient to achieve a maximal AUC₍₀₋ₜₐᵤ₎ of [−]-β-DHTBZ of about 5 hr*ng/ml in a patient who is not a CYP2D6 poor metabolizer or of about 32.5 hr*ng/ml in a patient who is a CYP2D6 poor metabolizer.

In certain embodiments, the VMAT2 inhibitor is administered in an amount sufficient to achieve a maximal AUC₍₀₋ₜₐᵤ₎ of [−]-β-DHTBZ of about 3.4 hr*ng/ml in a patient who is not a CYP2D6 poor metabolizer or of about 22 hr*ng/ml in a patient who is a CYP2D6 poor metabolizer.

Also provided is a method of treating a neurological or psychiatric disease or disorder in a patient in need thereof who is a CYP2D6 poor metabolizer, comprising:
administering to the patient in need thereof a vesicular monoamine transporter 2 (VMAT2) inhibitor via a titration scheme that comprises the up-titration of the VMAT2 inhibitor from an initial dose in increments not exceeding about 6 mg/day at about weekly intervals until a maintenance dose is administered, wherein the maintenance dose does not exceed a total daily dose of about 30 mg of the VMAT2 inhibitor,
wherein the VMAT2 inhibitor is (RR,SS)-1, 3, 4, 6, 7, 11b-hexahydro-9,10-di(methoxy-d3)-3-(2-methylpropyl)-2H-benzo[a]quinolizin-2-one (deutetrabenazine) or an enantiomer or pharmaceutically acceptable salt thereof.

In some embodiments, the initial dose is about 3 mg once daily. In some embodiments, the initial dose is about 3 mg twice daily. In some embodiments, the increment is about 6 mg/day.

In some embodiments, the initial dose is about 6 mg once daily. In some embodiments, the initial dose is about 6 mg twice daily. In some embodiments, the increment is about 3 mg/day.

In some embodiments, the initial dose is about 5 mg once daily. In some embodiments, the initial dose is about 5 mg twice daily. In some embodiments, the increment is about 5 mg/day.

In some embodiments, the initial dose is about 6 mg once daily. In some embodiments, the initial dose is about 6 mg twice daily. In some embodiments, the increment is about 6 mg/day.

In some embodiments, the VMAT2 inhibitor is administered via a titration scheme that comprises the up-titration of VMAT2 inhibitor at about weekly intervals until a maintenance dose is administered.

In some embodiments, the up-titration scheme is modified based on tolerability. In some embodiments, a patient is not able to tolerate the treatment regimen and the dose is reduced or administration is suspended.

In some embodiments, the maximum permitted dose is not reached because the patient has achieved satisfactory reduction of chorea associated with Huntington's disease. In some embodiments, the maximum permitted dose is not reached because the patient has achieved satisfactory control of tardive dyskinesia.

In some embodiments, the efficacy of treating chorea associated with Huntington's disease is measured by the Total Maximal Chorea Score. In some embodiments, the efficacy of treating tardive dyskinesia is measured by the Abnormal Involuntary Movement Scale.

In some embodiments, the goal of titration is to achieve an optimal level of disease control in which the patient is tolerating the treatment regimen or until the maximum permitted dose is reached. If a patient is not able to tolerate the treatment regimen, in some embodiments, the dose may be further reduced or administration may be suspended.

In certain embodiments, the maintenance dose of the VMAT2 inhibitor for a patient who is a CYP2D6 poor metabolizer is a total daily dosage of deutetrabenazine that does not exceed about 30 mg per day. In some embodiments, the total daily dose is administered in a maximum single dose of about 15 mg twice per day. In some embodiments, the total daily dose is administered once daily.

In certain embodiments, the maintenance dose of the VMAT2 inhibitor for a patient who is a CYP2D6 poor metabolizer is a total daily dosage of deutetrabenazine that does not exceed about 25 mg. In some embodiments, the total daily dose is administered in a maximum single dose of about 12.5 mg twice per day. In some embodiments, the total daily dose is administered once daily.

In certain embodiments, the maintenance dose of the VMAT2 inhibitor for a patient who is a CYP2D6 poor metabolizer is a total daily dosage of deutetrabenazine that does not exceed about 24 mg. In some embodiments, the total daily dose is administered in a maximum single dose of about 12 mg twice per day. In some embodiments, the total daily dose is administered once daily.

In certain embodiments, the maintenance dose of the VMAT2 inhibitor for a patient who is a CYP2D6 poor metabolizer is a total daily dosage of deutetrabenazine that does not exceed about 20 mg. In some embodiments, the total daily dose is administered in a maximum single dose of about 10 mg twice per day. In some embodiments, the total daily dose is administered once daily.

In certain embodiments, the maintenance dose of the VMAT2 inhibitor for a patient who is a CYP2D6 poor metabolizer is a total daily dosage of deutetrabenazine that does not exceed about 18 mg. In some embodiments, the total daily dose is administered in a maximum single dose of about 9 mg twice per day. In some embodiments, the maximum daily dose is administered once daily.

In certain embodiments, the maintenance dose of the VMAT2 inhibitor for a patient who is a CYP2D6 poor metabolizer is a total daily dosage of deutetrabenazine that does not exceed about 15 mg. In some embodiments, the total daily dose is administered in a maximum single dose of about 7.5 mg twice per day. In some embodiments, the total daily dose is administered once daily.

In certain embodiments, the maintenance dose of the VMAT2 inhibitor for a patient who is a CYP2D6 poor metabolizer is a total daily dosage of deutetrabenazine that does not exceed about 12 mg. In some embodiments, the total daily dose is administered in a maximum single dose of about 6 mg twice per day. In some embodiments, the total daily dose is administered once daily.

In certain embodiments, the maintenance dose of the VMAT2 inhibitor for a patient who is a CYP2D6 poor metabolizer is a total daily dosage of deutetrabenazine that does not exceed about 10 mg. In some embodiments, the total daily dose is administered in a maximum single dose of about 5 mg twice per day. In some embodiments, the total daily dose is administered once daily.

In certain embodiments, the maintenance dose of the VMAT2 inhibitor for a patient who is a CYP2D6 poor metabolizer is a total daily dosage of deutetrabenazine that does not exceed about 6 mg. In some embodiments, the total daily dose is administered in a maximum single dose of about 3 mg twice per day. In some embodiments, the total daily dose is administered once daily.

In certain embodiments, the maintenance dose of the VMAT2 inhibitor for a patient who is a CYP2D6 poor metabolizer is a total daily dosage of deutetrabenazine that does not exceed about 5 mg. In some embodiments, the total daily dose is administered once daily.

In certain embodiments, the VMAT2 inhibitor, is administered via a titration scheme that comprises administering a first dose for a period of one week; further increasing the dose by an amount equal to an incremental value; and determining whether the subject tolerates the further increased dose; wherein the cycle is repeated so long as the subject tolerates the further increased dose, wherein the incremental value at each cycle repetition is the same or different; and wherein if the subject does not tolerate the further increased dose, the modified dose for the subject is equal to the difference between the further increased dose and the incremental value for the last cycle repetition. In some embodiments, the first dose is about 6 mg once daily and the incremental value is about 6 mg per day.

In some embodiments, increasing the dose comprises increasing the frequency of dosing. In some embodiments, the first dose is about 6 mg once daily and the increased dose is about 6 mg twice daily.

In some embodiments, the titration scheme comprises the following:
about 6 mg once daily for a week;
about 6 mg twice daily for a week;
about 9 mg twice daily for a week;
about 12 mg twice daily for a week; and
optionally continued increase of an additional about 3 mg twice daily per week until the maintenance dose is administered.

In some embodiments, the titration scheme comprises the following:
about 5 mg once daily for a week;
about 5 mg twice daily for a week;
about 8 mg twice daily for a week;
about 10 mg twice daily for a week; and
optionally continued increase of an additional about 3 mg twice daily per week until the maintenance dose is administered.

In some embodiments, total daily dosages of 12 mg or above are administered in two divided doses.

In some embodiments, total daily dosages of 12 mg or above are administered in one single dose.

In certain embodiments, the method further comprises determining whether the patient is a CYP2D6 poor metabolizer.

In certain embodiments, the patient has a CYP2D6 poor metabolizer genotype.

In certain embodiments, the CYP2D6 poor metabolizer genotype is chosen from the CYP2D6G1846A genotype or the CYP2D6C100T genotype.

In certain embodiments, the CYP2D6 poor metabolizer genotype is the CYP2D6G1846A (AA) genotype or the CYP2D6G1846A (AG) genotype.

In certain embodiments, the CYP2D6 poor metabolizer genotype is the CYP2D6G1846A (AA) genotype.

In certain embodiments, the CYP2D6 poor metabolizer genotype is the CYP2D6C100T (TT) genotype or the CYP2D6C100T (CT) genotype.

In certain embodiments, the CYP2D6 poor metabolizer genotype is the CYP2D6C100T (TT) genotype.

In some embodiments, the neurological or psychiatric disease or disorder is a hyperkinetic movement disorder, mood disorder, bipolar disorder, schizophrenia, schizoaffective disorder, mania in mood disorder, depression in mood disorder, treatment-refractory obsessive compulsive disorder, neurological dysfunction associated with Lesch-Nyhan syndrome, agitation associated with Alzheimer's disease, Fragile X syndrome or Fragile X-associated tremor-ataxia syndrome, autism spectrum disorder, Rett syndrome, or chorea-acanthocytosis.

In some embodiments, the neurological or psychiatric disease or disorder is a hyperkinetic movement disorder.

In some embodiments, the hyperkinetic movement disorder is tardive dyskinesia.

In some embodiments, the hyperkinetic movement disorder is Tourette's syndrome.

In some embodiments, the hyperkinetic movement disorder is Huntington's disease.

In some embodiments, the hyperkinetic movement disorder is tics.

In some embodiments, the hyperkinetic movement disorder is chorea. In a further embodiment, the hyperkinetic movement disorder is chorea associated with Huntington's disease.

In some embodiments, the hyperkinetic movement disorder is ataxia, chorea, dystonia, Huntington's disease, myoclonus, restless leg syndrome, or tremors.

In certain embodiments, the maximum daily dose is less than the amount that is administered to a patient who is not a CYP2D6 poor metabolizer.

In certain embodiments, the maximum daily dose of the VMAT2 inhibitor is 10-90% less than the amount that would be administered to a patient who is not a CYP2D6 poor metabolizer.

In certain embodiments, the maximum daily dose of the VMAT2 inhibitor is 20-80% less than the amount that would be administered to a patient who is not a CYP2D6 poor metabolizer.

In certain embodiments, the maximum daily dose of the VMAT2 inhibitor is 30-70% less than the amount that would be administered to a patient who is not a CYP2D6 poor metabolizer.

In certain embodiments, the maximum daily dose of the VMAT2 inhibitor is 40-60% less than the amount that would be administered to a patient who is not a CYP2D6 poor metabolizer.

In certain embodiments, the maximum daily dose of the VMAT2 inhibitor is about 50% less than the amount that would be administered to a patient who is not a CYP2D6 poor metabolizer.

For example, where the dosage administered to a patient who is not a CYP2D6 poor metabolizer is 48 mg per day, an individual who is a CYP2D6 poor metabolizer may receive a reduced dosage of a maximum of about 5 to about 30 mg/day, such as a maximum of about 18 mg per day per day. Likewise, where the dosage administered to a patient who is not a CYP2D6 poor metabolizer is a maximum single dose of 24 mg, an individual who is a CYP2D6 poor metabolizer may receive a reduced dosage of about 6 to about 12 mg, such as a maximum single dose of about 9 mg.

In some embodiments, the total daily dose does not exceed about 30 mg with a maximum single dose of about 15 mg. In some embodiments, the total daily dose is administered once daily.

In some embodiments, the total daily dose does not exceed about 27 mg with a maximum single dose of about 13.5 mg. In some embodiments, the total daily dose is administered once daily.

In some embodiments, the total daily dose does not exceed about 24 mg with a maximum single dose of about 12 mg. In some embodiments, the total daily dose is administered once daily.

In some embodiments, the total daily dose does not exceed about 21 mg with a maximum single dose of about 10.5 mg. In some embodiments, the total daily dose is administered once daily.

In some embodiments, the total daily dose does not exceed about 20 mg with a maximum single dose of about 10 mg. In some embodiments, the total daily dose is administered once daily.

In some embodiments, the total daily dose does not exceed about 18 mg with a maximum single dose of about 9 mg. In some embodiments, the total daily dose is administered once daily.

In some embodiments, the total daily dose does not exceed about 15 mg with a maximum single dose of about 7.5 mg. In some embodiments, the total daily dose is administered once daily.

In some embodiments, the total daily dose does not exceed about 12 mg with a maximum single dose of about 6 mg. In some embodiments, the total daily dose is administered once daily.

In some embodiments, the total daily dose does not exceed about 10 mg with a maximum single dose of about 5 mg. In some embodiments, the total daily dose is administered once daily.

In some embodiments, the total daily dose does not exceed about 9 mg with a maximum single dose of about 5 mg. In some embodiments, the total daily dose is administered once daily.

In some embodiments, the total daily dose does not exceed about 6 mg with a maximum single dose of about 3 mg. In some embodiments, the total daily dose is administered once daily.

In some embodiments, the method further comprises monitoring the patient for one or more exposure-related adverse reactions.

In some embodiments, the method further comprises reducing the amount of the VMAT2 inhibitor. In some embodiments, the method further comprises reducing the amount of the VMAT2 inhibitor based on the patient's ability to tolerate one or more exposure-related adverse reactions.

In some embodiments, the method further comprises informing the patient or a medical care worker that administration of the VMAT2 inhibitor to a patient who is a CYP2D6 poor metabolizer may result in increased risk of one or more exposure-related adverse reactions.

In some embodiments, the method further comprises informing the patient or a medical care worker that administration of the VMAT2 inhibitor to a patient who is a CYP2D6 poor metabolizer may prolong the patient's QT interval.

In some embodiments, the one or more exposure-related adverse reactions is chosen from somnolence and sedation.

In some embodiments, the one or more exposure-related adverse reactions is chosen from QTc prolongation, neuroleptic malignant syndrome (NMS), akathisia, agitation, restlessness, parkinsonism, sedation, somnolence, hyperprolactinemia, and binding to melanin-containing tissues.

In some embodiments, the neurological or psychiatric disease or disorder is chorea associated with Huntington's disease is one or more exposure-related adverse reactions is chosen from diarrhea, dry mouth, fatigue, sedation/somnolence, depression and suicidality, parkinsonism, akathisia, restlessness, and cognitive decline, urinary tract infection, insomnia, anxiety, constipation, and contusion.

In some embodiments, the neurological or psychiatric disease or disorder is tardive dyskinesia is one or more exposure-related adverse reactions is chosen from nasopharyngitis, insomnia, depression/dysthymic disorder, and akathisia/agitation/restlessness.

In some embodiments, the VMAT2 inhibitor is administered orally.

In some embodiments, the VMAT2 inhibitor is administered in the form of a tablet or capsule.

In any one of the above-mentioned embodiments, the VMAT2 inhibitor is deutetrabenazine free base.

Examples of embodiments of the present disclosure are provided in the following examples. The following examples are presented only by way of illustration and to assist one of ordinary skill in using the disclosure. The examples are not intended in any way to otherwise limit the scope of the disclosure.

### EXAMPLES

### Example 1: Crossover Study to Determine the Single-Dose Pharmacokinetics of Deutetrabenazine Metabolites in Healthy Subjects

A phase 1, open-label, 2-sequence crossover study was conducted to evaluate the HTBZ PK profile following administration of valbenazine (40 mg) or deutetrabenazine (24 mg) to a total of 18 healthy male and female subjects.

Subjects received a single dose of valbenazine 40 mg on Day 1 and a single dose of deutetrabenazine 24 mg on Day 16; or received a single dose of deutetrabenazine 24 mg on Day 1 and a single dose of valbenazine 40 mg on Day 16. Valbenazine 40 mg was administered as one 40 mg capsule and deutetrabenazine 24 mg was administered as two 12 mg tablets. Study drug was administered at approximately 0800 hours, approximately 30 minutes after the start of a standard breakfast.

Valbenazine parent compound and its [+]-α-HTBZ metabolite (NBI-98782), and each of the 4 deutetrabenazine metabolites ([+]-α-DHTBZ, [−]-α-DHTBZ, [+]-β-DHTBZ, and [−]-β-DHTBZ) were quantified using validated bioanalytical methods. PK parameters were determined using non-compartmental analysis with Phoenix WinNonlin 8.3 software. The in vitro pharmacology of the deuterated HTBZ metabolites was assessed using competitive radioligand binding assays.

Only the [+]-DHTBZ isomers were shown to inhibit VMAT2 activity in vitro. [−]-α-DHTBZ, which is inactive as a VMAT2 inhibitor, was the most abundant metabolite observed after deutetrabenazine administration, accounting for 66% of total DHTBZ exposure. 69% of total AUC of all deutetrabenazine metabolites are inactive metabolites [−]-α-DHTBZ and [−]-β-DHTBZ.

[+]-β-DHTBZ was the most abundant circulating active DHTBZ metabolite following deutetrabenazine administration, accounting for 29% of total circulating DHTBZ metabolites. The most abundant circulating isoforms are inactive [−]-α-DHTBZ and [−]-β-DHTBZ (69% of total AUC) [+]-α-DHTBZ and [−]-β-DHTBZ were minor metabolites after deutetrabenazine administration, accounting for only 2% and 3%, respectively, of circulating HTBZ metabolites. The mean half-life of [+]-β-DHTBZ, the predominant active deutetrabenazine metabolite is 7.7 hours. Peak plasma concentrations (Cₘₐₓ) of deuterated [+/−] α-DHTBZ and [+/−] β-DHTBZ are reached within 2 to 6 hours after dosing.

The half-life of total (α+β)-DHTBZ from deutetrabenazine is approximately 9 to 10 hours. The mean half life for [−]-α-DHTBZ, [+]-α-DHTBZ and the [−]-β-DHTBZ and [+]-β-DHTBZ metabolites of AUSTEDO are approximately 12, 6, 8, and 5 hours, respectively.

| | | | AUC |
|---|---|---|---|
| Analyte | Cmax (ng/mL) | AUC (hr*ng/ml) | (% of Total) |
| [+]-α-DHTBZ | 1.2 (25) | 11 (42) | 2 |
| [−]-α-DHTBZ | 32.2 (22) | 396 (30) | 66 |
| [+]-β-DHTBZ | 20.4 (35) | 176 (57) | 29 |
| [−]-β-DHTBZ | 1.7 (44) | 15.8 (63) | 3 |

*The numbers in the parentheses reflect the % CV (coefficient of variation) for each estimate.

### Example 2: VMAT2 Receptor Binding of Deutetrabenazine and Valbenazine Metabolites

Methods employed in this study have been adapted from the scientific literature to maximize reliability and reproducibility (Eur J Pharmacol. 186(1): 95-104; Life Sci. 69(19): 2311-2317). Reference standards were run as an integral part of each assay to ensure the validity of the results obtained. Assays were performed under conditions described in the accompanying “Methods” section of this report. Where presented, IC50 values were determined by a non-linear, least squares regression analysis using MathIQ™ (ID Business Solutions Ltd., UK). Where inhibition constants (Ki) are presented, the Ki values were calculated using the equation of Cheng and Prusoff (Cheng, Y., Prusoff, W.H., Biochem. Pharmacol. 22:3099-3108, 1973) using the observed IC50 of the tested compound, the concentration of radioligand employed in the assay, and the historical values for the KD of the ligand (obtained experimentally at Eurofins Panlabs, Inc.). Where presented, the Hill coefficient (nH), defining the slope of the competitive binding curve, was calculated using MathIQ™. Hill coefficients significantly different than 1.0, may suggest that the binding displacement does not follow the laws of mass action with a single binding site. Where IC50, Ki, and/or nH data are presented without Standard Error of the Mean (SEM), data are insufficient to be quantitative, and the values presented (Ki, IC50, nH) should be interpreted with caution. The results are summarized in the table below.

VMAT2 Receptor Binding Affinity, Ki (nM)

| | | | | Valbenazine |
|---|---|---|---|---|
| Deutetrabenazine Metabolites | | | | Metabolite |
| [−]-β- | [−]-α- | [+]-β- | [+]-α- | [+]-α- |
| DHTBZ | DHTBZ | DHTBZ | DHTBZ | HTBZ |
| 1220 | >3000 | 17.1 | 1.5 | 1.4 |

The primary active metabolite for deutetrabenazine ([+]-β-DHTBZ) is different from valbenazine ([+]-α-HTBZ). Determination of the effect(s) of intrinsic and extrinsic variables on deutetrabenazine safety and efficacy profile should incorporate assessment of the effects on the individual primary circulating HTBZ metabolites, [−]-α-DHTBZ and [+]-β-DHTBZ. [−]-α-DHTBZ is most abundant metabolite observed. Specifically, [−]-α-DHTBZ and [−]-β-DHTBZ have a potential for off-target effects on 5-HT7 and D2 receptors.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of treating a neurological or psychiatric disease or disorder in a patient in need thereof, comprising:
administering a maximum total daily dose of between about 6 mg/day and about 30 mg/day of a vesicular monoamine transporter 2 (VMAT2) inhibitor to the patient in need thereof, wherein the patient a CYP2D6 poor metabolizer,
wherein the VMAT2 inhibitor is (RR,SS)-1, 3, 4, 6, 7, 11b-hexahydro-9,10-di(methoxy-d3)-3-(2-methylpropyl)-2H-benzo[a]quinolizin-2-one (deutetrabenazine),
wherein the administering occurs outside of a clinical trial setting.

2. A method of treating a neurological or psychiatric disease or disorder in a patient in need thereof, comprising:
administering to the patient a maximum total daily dose of deutetrabenazine, that is between about 6 mg/day and about 30 mg/day if, and because, the patient is a CYP2D6 poor metabolizer,
wherein the administering occurs outside of a clinical trial setting.

3. A method of treating a neurological or psychiatric disease or disorder in a patient in need thereof, wherein the patient is a CYP2D6 poor metabolizer comprising:
administering to the patient a maximum total daily dose of between 6 mg/day and about 30 mg/day of a vesicular monoamine transporter 2 (VMAT2) inhibitor,
wherein the VMAT2 inhibitor is deutetrabenazine,
monitoring the concentration of [−]-α-DHTBZ and/or [−]-β-DHTBZ in the patient's blood; and
reducing the amount of the VMAT2 inhibitor being administered when the [−]-α-DHTBZ and/or [−]-β-DHTBZ exposure in the patient's blood is increased as compared with the [−]-α-DHTBZ and/or [−]-β-DHTBZ level in a patient who is administered the VMAT2 inhibitor alone,
wherein the administering occurs outside of a clinical trial setting.

4. The method of claim 3, wherein the [−]-α-DHTBZ and [−]-β-DHTBZ exposures are measured as the area under the plasma concentration versus time curve from 0 hours extrapolated to infinity or measured as the maximum observed blood plasma concentration (Cₘₐₓ) at the time of maximum plasma concentration (tₘₐₓ).

5. The method of claim 3 or 4, wherein the increased [−]-α-DHTBZ and/or [−]-β-DHTBZ exposure increases the risk of one or more exposure-related adverse reactions.

6. A method of treating a neurological or psychiatric disease or disorder in a patient in need thereof, comprising:
administering about 48 mg/day of a vesicular monoamine transporter 2 (VMAT2) inhibitor to the patient in need thereof, wherein the VMAT2 inhibitor is deutetrabenazine,
subsequently determining that the patient is a CYP2D6 poor metabolizer and
administering a maximum total daily dose of between about 6 mg/day and about 30 mg/day of a vesicular monoamine transporter 2 (VMAT2) inhibitor to the patient in need thereof,
wherein the administering occurs outside of a clinical trial setting.

7. A method of decreasing a risk of QT prolongation in a patient being treated for a neurological or psychiatric disease or disorder with deutetrabenazine, comprising:
administering to the patient a maximum total daily dose of deutetrabenazine that is between about 6 mg/day and about 30 mg/day if, and because, the patient is a CYP2D6 poor metabolizer,
wherein the administering occurs outside of a clinical trial setting.

8. A method of reducing off-target activity and increasing on-target activity in a patient being treated for a neurological or psychiatric disease or disorder with deutetrabenazine, comprising:
administering to the patient a maximum total daily dose of deutetrabenazine that is between about 6 mg/day and about 30 mg/day if, and because, the patient is a CYP2D6 poor metabolizer,
wherein the administering occurs outside of a clinical trial setting.

9. The method of claim 8, wherein the off-target effects are caused by (−)-α-3-isobutyl-9,10-di(methoxy-d3)-1,3,4,6,7,11b-hexahydro-2H-pyrido[2,1-a]isoquinolin-2-ol ([−]-α-DHTBZ) and (−)-β-3-isobutyl-9,10-di(methoxy-d3)-1,3,4,6,7,11b-hexahydro-2H-pyrido[2,1-a]isoquinolin-2-ol ([−]-β-DHTBZ).

10. The method of claim 8, wherein the [−]-α-DHTBZ and [−]-β-DHTBZ activate an off-target receptor chosen from serotonin 5-HT7 receptor and dopamine D2 receptors.

11. The method of claim 8, wherein the on-target effects are caused by (+)-β-3-isobutyl-9,10-di(methoxy-d3)-1,3,4,6,7,11b-hexahydro-2H-pyrido[2,1-a]isoquinolin-2-ol ([+]-β-DHTBZ).

12. The method of any one of the preceding claims, wherein the patient has a CYP2D6 poor metabolizer genotype.

13. The method of claim 12, wherein the CYP2D6 poor metabolizer genotype is chosen from the CYP2D6G1846A genotype or the CYP2D6C100T genotype.

14. The method of claim 13, wherein the CYP2D6 poor metabolizer genotype is the CYP2D6G1846A (AA) genotype or the CYP2D6G1846A (AG) genotype.

15. The method of claim 14, wherein the CYP2D6 poor metabolizer genotype is the CYP2D6G1846A (AA) genotype.

16. The method of claim 13, wherein the CYP2D6 poor metabolizer genotype is the CYP2D6C100T (TT) genotype or the CYP2D6C100T (CT) genotype.

17. The method of claim 16, wherein the CYP2D6 poor metabolizer genotype is the CYP2D6C100T (TT) genotype.

18. The method of any one of the preceding claims, wherein the neurological or psychiatric disease or disorder is a hyperkinetic movement disorder, mood disorder, bipolar disorder, schizophrenia, schizoaffective disorder, mania in mood disorder, depression in mood disorder, treatment-refractory obsessive compulsive disorder, neurological dysfunction associated with Lesch-Nyhan syndrome, agitation associated with Alzheimer's disease, Fragile X syndrome or Fragile X-associated tremor-ataxia syndrome, autism spectrum disorder, Rett syndrome, or chorea-acanthocytosis.

19. The method of claim 18, wherein the neurological or psychiatric disease or disorder is a hyperkinetic movement disorder.

20. The method of claim 18, wherein the hyperkinetic movement disorder is tardive dyskinesia.

21. The method of claim 18, wherein the hyperkinetic movement disorder is Tourette's syndrome.

22. The method of claim 18, wherein the hyperkinetic movement disorder is Huntington's disease.

23. The method of claim 18, wherein the hyperkinetic movement disorder is tics.

24. The method of claim 18, wherein the hyperkinetic movement disorder is chorea associated with Huntington's disease.

25. The method of claim 18, wherein the hyperkinetic movement disorder is ataxia, chorea, dystonia, Huntington's disease, myoclonus, restless leg syndrome, or tremors.

26. The method of any one of the preceding claims, wherein the maximum daily dose is about 24 mg.

27. The method of claim 26, wherein the maximum daily dose is administered in a maximum single dose of about 12 mg.

28. The method of any one of claims 1 to 25, wherein the maximum daily dose is about 20 mg.

29. The method of claim 28, wherein the maximum daily dose is administered in a maximum single dose of about 10 mg.

30. The method of any one of claims 1 to 25, wherein the maximum daily dose is about 18 mg.

31. The method of claim 30, wherein the maximum daily dose is administered in a maximum single dose of about 9 mg.

32. The method of any one of claims 1 to 25, wherein the maximum daily dose is about 12 mg.

33. The method of claim 32, wherein the maximum daily dose is administered in a maximum single dose of about 6 mg.

34. The method of any one of the preceding claims, further comprising monitoring the patient for one or more exposure-related adverse reactions.

35. The method of any one of the preceding claims, further comprising reducing the amount of the VMAT2 inhibitor based on the patient's ability to tolerate one or more exposure-related adverse reactions.

36. The method of any one of the preceding claims, further comprising informing the patient or a medical care worker that administration of the VMAT2 inhibitor to a patient who is a CYP2D6 poor metabolizer may result in increased risk of one or more exposure-related adverse reactions.

37. The method of any one of the preceding claims, further comprising informing the patient or a medical care worker that administration of the VMAT2 inhibitor to a patient who is a CYP2D6 poor metabolizer may prolong the patient's QT interval.

38. The method of any one of claims 34 to 37, wherein the one or more exposure-related adverse reactions is chosen from somnolence and sedation.

39. The method of any one of claims 34 to 37, wherein the one or more exposure-related adverse reactions is chosen from QTc prolongation, neuroleptic malignant syndrome (NMS), akathisia, agitation, restlessness, parkinsonism, sedation, somnolence, hyperprolactinemia, and binding to melanin-containing tissues.

40. The method of any one of claims 34 to 37, wherein the neurological or psychiatric disease or disorder is chorea associated with Huntington's disease is one or more exposure-related adverse reactions is chosen from diarrhea, dry mouth, fatigue, sedation/somnolence, depression and suicidality, parkinsonism, akathisia, restlessness, and cognitive decline, urinary tract infection, insomnia, anxiety, constipation, and contusion.

41. The method of any one of claims 34 to 37, wherein the neurological or psychiatric disease or disorder is tardive dyskinesia is one or more exposure-related adverse reactions is chosen from nasopharyngitis, insomnia, depression/dysthymic disorder, and Akathisia/Agitation/Restlessness.

42. The method of any one of the preceding claims, wherein the VMAT2 inhibitor is administered orally.

43. The method of any one of the preceding claims, wherein the VMAT2 inhibitor is administered in the form of a tablet or capsule.
